(19) United States
(12) Patent Application Publication
(10) Pub. No.: US 2025/0078990 A1
(43) Pub. Date: Mar. 6, 2025

(54) VIRTUAL REALITY TRAINING SYSTEM FOR INVENTORY STORAGE LOCATIONS IN A HEALTHCARE FACILITY

(71) Applicant: Hill-Rom Services, Inc., Batesville, IN (US)

(72) Inventors: Stacie Lynn Brough, Onondaga, NY (US); Kathryn Piston, Syracuse, NY (US); Autumn S. Thompson, Philadelphia, PA (US)

(21) Appl. No.: 18/790,783

(22) Filed: Jul. 31, 2024

Related U.S. Application Data
(60) Provisional application No. 63/535,983, filed on Aug. 31, 2023.

(51) Int. Cl. G16H 40/20; G06T 19/00; G16H 40/40

(52) U.S. Cl. CPC G16H 40/20; G06T 19/00; G16H 40/40

(57) ABSTRACT

A virtual reality training system for a healthcare facility is provided. The system includes a plurality of healthcare devices and a real time locating system for locating each of the plurality of devices within the healthcare facility. A learning and management system stores device data and facility data. A virtual reality application is configured for training providers in the healthcare facility based on the device data and the facility data.

### CROSS-REFERENCE TO RELATED APPLICATIONS

This application claims priority under 35 U.S.C. § 119(e) to U.S. Provisional Application No. 63/535,983, filed Aug. 31, 2023, which is expressly incorporated by reference herein.

### BACKGROUND

The present disclosure relates to a training in healthcare facility and, in particular, to a virtual reality training system for a healthcare facility.

With the rise in travel nursing and float staff, adjusting to different healthcare facility layouts and workflows can be difficult for nursing staff. Many healthcare facilities have attempted to solve this problem by standardizing across the healthcare system, but this can be a costly undertaking. Untrained users can create patient safety issues through improper use of devices. Furthermore, new hires do not always get to experience the official training during the deployment of different devices. Previous trainings could be recorded and converted into personalized virtual reality training for the new hire to experience.

### SUMMARY

The present disclosure includes one or more of the features recited in the appended claims and/or the following features which, alone or in any combination, may comprise patentable subject matter.

According to a first aspect of the disclosed embodiments, a virtual reality training system for a healthcare facility includes a real time locating system for locating each of a plurality of healthcare devices within a healthcare facility. The plurality of healthcare devices belong within a predetermined category of healthcare devices. A learning and management system is provided for storing device data related to a location of each of the plurality of healthcare devices within the predetermined category. The learning and management system further stores facility data including a map of the healthcare facility. A virtual reality application is configured for training providers in the healthcare facility. The virtual reality application is configured to display images of the healthcare facility based on the facility data. The images of the healthcare facility include representations of each of the plurality of healthcare devices within the predetermined category at the respective location in the healthcare facility based on the device data. A provider utilizes the virtual reality application to acclimate themselves to the location of each of the plurality of healthcare devices within the predetermined category.

In some embodiments of the first aspect, the predetermined category of healthcare devices may include healthcare devices related to a role of the provider in the healthcare facility. The predetermined category of healthcare devices may include healthcare devices in storage at the healthcare facility. The predetermined category of healthcare devices may include healthcare devices related to a type of training module. The virtual reality application is operated on a virtual reality headset worn by the provider. The provider may utilize the virtual reality application to acclimate themselves to predetermined locations within the healthcare facility based on a role of the provider within the healthcare facility. The system may track a progress of the provider in acclimating themselves to the predetermined locations. The system may approve the provider for the role based on the progress of the provider. The real time locating system may update the location of one of the plurality of healthcare devices in response to the one of the plurality of healthcare devices being moved. The device data related to the location of each of the plurality of healthcare devices may be updated in response to one of the plurality of healthcare devices being taken offline. The device data related to the location of each of the plurality of healthcare devices may be updated in response to a new healthcare device being implemented at the healthcare facility. The learning and management system may include a library of device training simulations to train the provider on operation of at least one of the plurality of healthcare devices using the virtual reality application. The provider's access to the at least one of the plurality of healthcare devices may be based on the device training simulations completed. The provider's access to the at least one of the plurality of healthcare devices may be limited based on the device training simulations completed. The provider may be granted full access to the at least one of the plurality of healthcare devices based on the device training simulations completed. The system may track the device training simulations completed by the provider. The provider may include a medical provider. The provider may include at least one of maintenance personnel, cleaning personnel, and administration personnel. The plurality of healthcare devices may include a plurality of medical devices. The plurality of healthcare devices may include a plurality of non-medical devices.

According to a second aspect of the disclosed embodiments, a method of training a provider at a healthcare facility includes building a catalog of a plurality of healthcare devices in the healthcare facility. The method also includes storing a library of device training simulations into a learning and management system. Each device simulation relates to one of the plurality of healthcare devices. The method also includes storing facility data including a map of the healthcare facility into the learning and management system. The method also includes providing access to the device simulations through a virtual reality application configured to display images of the healthcare facility based on the facility data. The images of the healthcare facility include representations of each of the plurality of healthcare devices. The method also includes assigning a list of healthcare devices of the plurality of healthcare devices to a provider based on a role of the provider. The method also includes tracking the provider's completion, with the virtual reality application, of the device simulations related to the list of healthcare devices assigned to the provider.

In some embodiments of the second aspect, the method may also include storing, in the learning and management system, device data related to a location of each of the plurality of healthcare devices within the healthcare facility based on data from a real time locating system. The images of the healthcare facility may include representations of each of the plurality of healthcare devices at the respective location in the healthcare facility based on the device data. The method may also include updating the location of one of the plurality of healthcare devices in response to the one of the plurality of healthcare devices being moved. The method may also include updating the device data related to the location of each of the plurality of healthcare devices in response to one of the plurality of healthcare devices being taken offline. The method may also include updating the device data related to the location of each of the plurality of healthcare devices in response to a new healthcare device being implemented at the healthcare facility. The virtual reality application is operated on a virtual reality headset worn by the provider. The provider may utilize the virtual reality application to acclimate themselves to the respective location of each of the plurality of healthcare devices in the healthcare facility. The provider may utilize the virtual reality application to acclimate themselves to predetermined locations within the healthcare facility based on the role of the provider within the healthcare facility. The method may also include tracking a progress of the provider in acclimating themselves to the predetermined locations. The method may also include approving the provider for the role based on the progress of the provider. The provider's access to the at least one of the plurality of healthcare devices may be based on the device training simulations completed. The method may also include limiting access to the at least one of the plurality of healthcare devices based on the device training simulations completed. The method may also include granting full access to the at least one of the plurality of healthcare devices based on the device training simulations completed. The method may also include tracking the device training simulations completed by the provider. The provider may include a healthcare provider. The provider may include at least one of maintenance personnel, cleaning personnel, and administration personnel. The plurality of healthcare devices may include a plurality of medical devices. The plurality of healthcare devices may include a plurality of non-medical devices.

Additional features, which alone or in combination with any other feature(s), such as those listed above and/or those listed in the claims, can comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 is a schematic diagram of a virtual reality training system for a healthcare facility;

FIG. 2 is a perspective view of an exemplary virtual reality headset and controller;

FIG. 3 is a perspective view of a screen positioned inside the virtual reality headset shown in FIG. 2 and used to generate a virtual reality environment;

FIG. 4 is an exemplary view of an image displayed on the screen shown in FIG. 3 and representing a healthcare facility environment;

FIG. 5 is an exemplary view of an image displayed on the screen shown in FIG. 3 and representing a healthcare device in the healthcare facility environment;

FIG. 6 is a perspective view of a user wearing the virtual reality headset and an image displayed on a screen in the virtual reality environment;

FIG. 7 is a flowchart of a method for completing training using the virtual reality training system shown in FIG. 1; and

FIG. 8 is a flow chart of a method for using the virtual reality training system shown in FIG. 1 to authenticate a user to operate medical devices in the healthcare facility.

### DETAILED DESCRIPTION

For the purposes of promoting an understanding of the principles of the disclosure, reference will now be made to a number of illustrative embodiments illustrated in the drawings and specific language will be used to describe the same.

The embodiments described herein provide a virtual reality training system 10 that healthcare staff can use to train on the location of items and different workflows via a digital real-time locating system (RTLS) map, when onboarding to a new unit or healthcare facility location. The system 10 is focused on creating a personalized training experience for staff based on the unique complexities of a healthcare facility, for example the physical layout of the facility, storage, or device locations. Data from RTLS is used to recreate the healthcare facility ecosystem, which the healthcare provider navigates via virtual reality, for example with a virtual reality headset. The healthcare provider uses the system 10 to complete different tasks and trainings, such as: acquiring different equipment, the use of personal protection equipment, or medication distribution, emergency exit and code protocol, tending to virtual patients, and how to work some of the healthcare facility technology and devices, such as the nurse call system. Additionally, RTLS asset tracking provides a live, up to date identification of where healthcare devices and equipment are stored. To supplement layout recreations, data from cameras in the healthcare facility creates a more realistic recreation of the healthcare facility floors. The data for the trainings, for example recordings from drills or device-specific training, are supplemented with recordings from previous trainings and converted into a virtual reality experience.

As the medical devices are part of the healthcare facility ecosystem, the connected devices have the ability to be smart devices by knowing if a user that is authenticating to use the device has been trained in its proper use. The connected device connects into a central connectivity system that performs the user authentication and can make a call to the healthcare facility learning & management system (LMS) to know if that user is trained. If not trained, the healthcare facility can configure the device to limit the functionality available to the user, make the device unavailable to untrained users, or flag the data so that it is used differently, ex. not to be used for clinical decision making, not to be charted in the EMR, or displayed as surveillance data only on visualization tools so that other clinical users know that data must be verified.

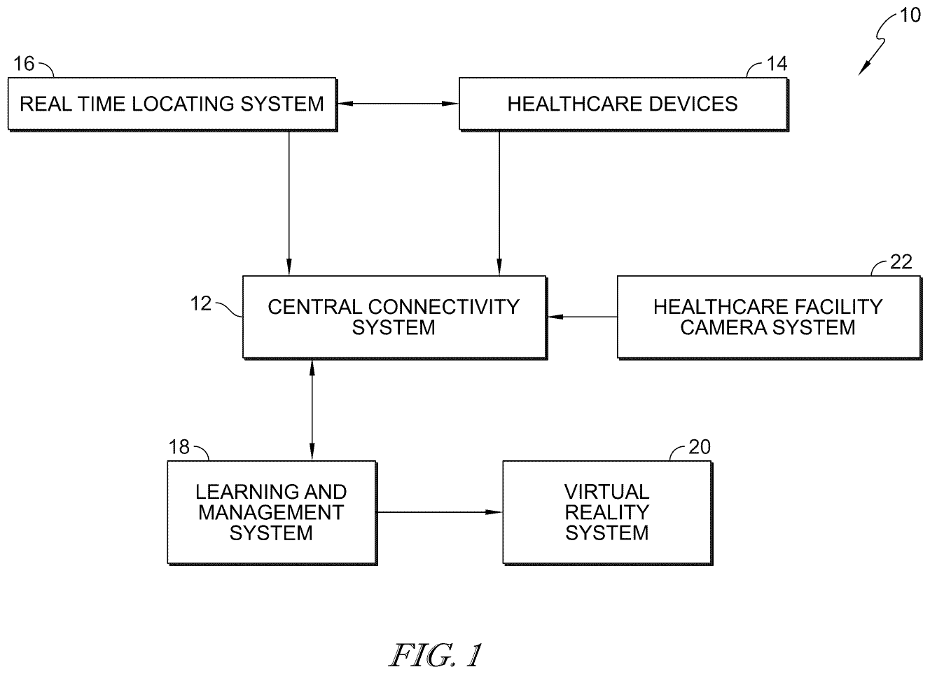

Referring now to FIG. 1, a virtual reality training system 10 for a healthcare facility includes a central connectivity system 12. As used herein, “healthcare facility” refers to any facility where healthcare is provided, for example a hospital, a nursing home, a doctor's office, or the like. It will be appreciated that although the embodiments described herein are described with respect to healthcare, in some embodiments, the system 10 is adaptable for use in other industries, for example manufacturing. The central connectivity system 12 provides a hub through which all other devices of the system 10 are connected and communicate. In an exemplary embodiment, the central connectivity system 12 includes at least one processor or microprocessor for manipulating data from the other devices of the system 10.

The system 10 includes a plurality of healthcare devices 14. In one embodiment, the healthcare devices 14 include medical devices. As such, a provider using such medical devices includes a healthcare provider. In other embodiments, the healthcare devices 14 include non-medical devices in the healthcare facility. As such, a provider using such non-medical devices includes maintenance personnel, cleaning personnel, administration personnel, or the like. In some embodiments, the plurality of healthcare devices 14 belong within a predetermined category of healthcare devices 14. In some embodiments, the predetermined category of healthcare devices 14 includes healthcare devices 14 related to a role of the provider in the healthcare facility. For example, the healthcare devices 14 are related to respiratory therapist or patient transporters, in some embodiments. In some embodiments, the predetermined category of healthcare devices 142 includes healthcare devices 14 in storage at the healthcare facility. In some embodiments, the predetermined category of healthcare devices 14 includes healthcare devices 14 related to a type of training module. For example, the healthcare devices 14 are related to code blue response or emergency room triage, in some embodiments. A location of each of the healthcare devices 14 is tracked by a real-time locating system (RTLS) 16 and communicated to the central connectivity system 12 as device data.

A learning and management system 18 stores the device data related to a location of each of the plurality of healthcare devices 14 within the healthcare facility. In some embodiments, the RTLS 16 updates the location of any one of the plurality of healthcare devices 14 in response to the one of the plurality of healthcare devices 14 being moved. For example, if a device 14 is moved from floor two to floor three, the RTLS 16 sends updated device data to the central connectivity system 12. In some embodiments, the device data related to the location of each of the plurality of healthcare devices 14 is updated in response to one of the plurality of healthcare devices being taken offline or removed from the healthcare facility. In another embodiment, the device data related to the location of each of the plurality of healthcare devices 14 is updated in response to a new healthcare device being implemented at the healthcare facility. The updated device data is stored in the learning and management system 18 so that the location of each device 14 is updated and tracked in real-time. The learning and management system 18 also stores facility data including a map of the healthcare facility. The facility data is specific to the healthcare facility and is updated along with changes to the floor plan of the facility, for example changes due to construction.

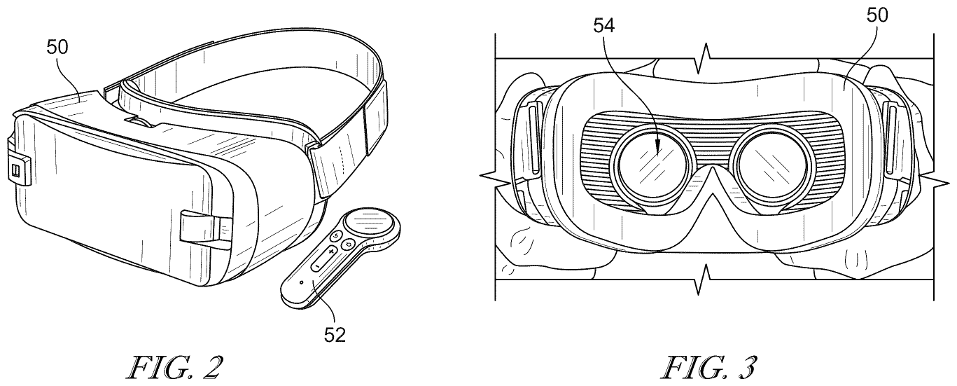

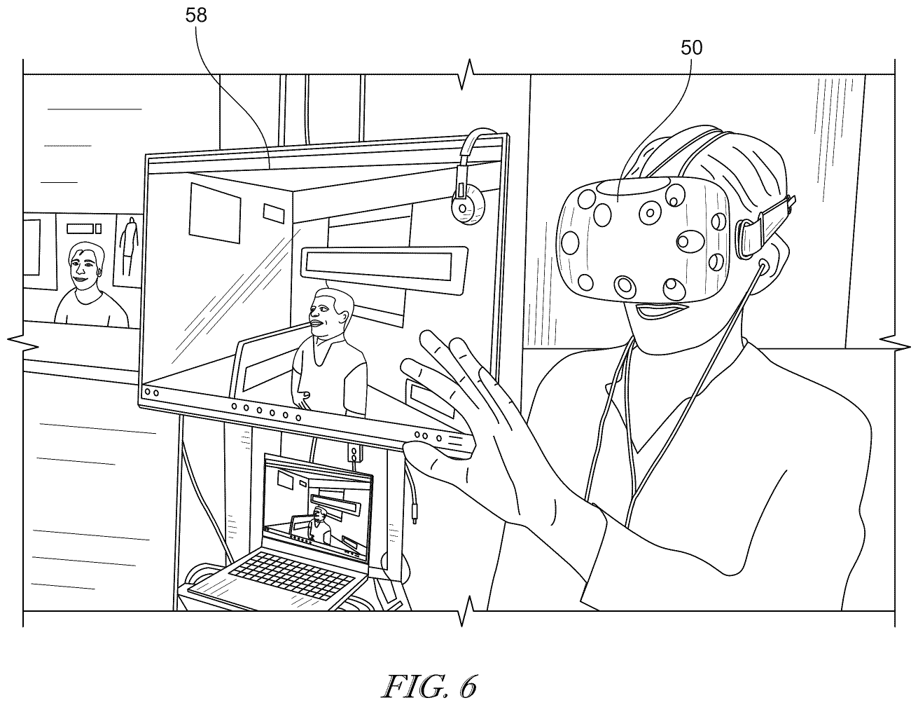

A virtual reality device 20 is configured for training providers in the healthcare facility. The virtual reality device 20 is configured to operate a virtual reality application. For example, the virtual reality device 20 is usable to train a provider on facility locations, as well as, device locations. In an exemplary embodiment, the virtual reality device 20 is used to train providers on how to use at least one of the devices 14. In an exemplary embodiment, the virtual reality device 20 includes a headset 50 and controller 52, as illustrated in FIG. 2. As illustrated in FIG. 3, the headset 50 includes a screen 54 that displays images to create a virtual environment of the healthcare facility. The controller 52 is useable to navigate the environment. It will be appreciated that in some embodiments, the virtual reality device 20 includes a computer having a monitor displaying the images to create the virtual environment of the healthcare facility. FIG. 6 illustrates a user wearing the headset 50 to navigate the virtual environment, which is displayed on a monitor 58.

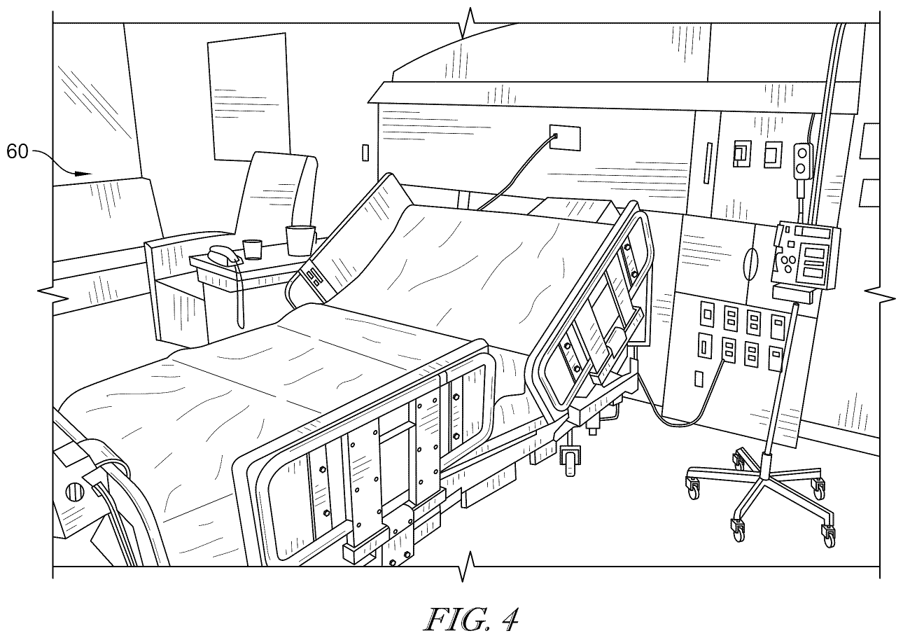

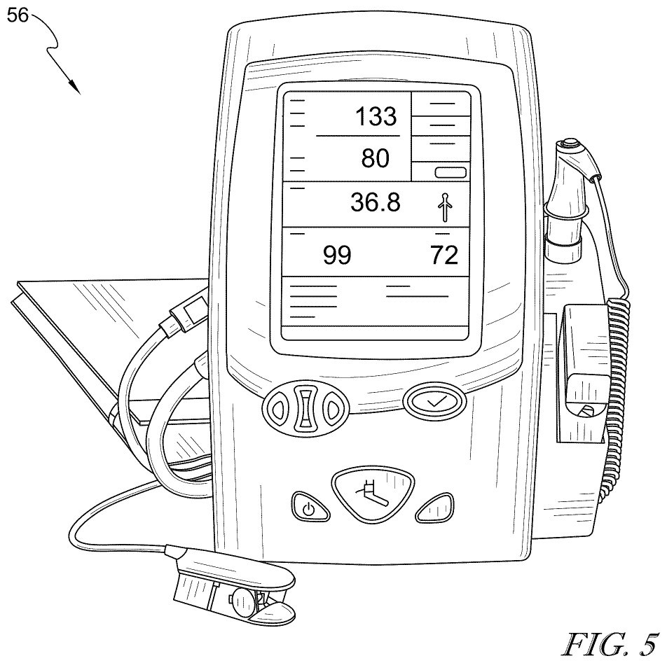

In an exemplary embodiment, the virtual reality device 20 displays images of the healthcare facility based on the facility data. That is, the virtual environment replicates the healthcare facility and rooms in the healthcare facility, as illustrated in FIG. 4, which displays a specific room 60 of the healthcare facility. Referring back to FIG. 1, in some embodiments, a camera system 22 of the healthcare facility is used to supplement the images and representations of the virtual environment. While navigating the virtual environment, for example with the controller 52, as user can virtually move from room to room within the healthcare facility. The images of the healthcare facility also include representations of each of the plurality of healthcare devices 14 at the respective location in the healthcare facility based on the device data. That is, if the RTLS 16 provides device data that a vital signs monitor 56 is located in room 202, a representation of the monitor 56 is shown in the virtual environment, as illustrated in FIG. 5, when the user virtually arrives at room 202. Accordingly, a provider utilizes the virtual reality device 20 to acclimate themselves to the location of each of the plurality of healthcare devices 14 in the healthcare facility. In one embodiment, the system 10 tracks a progress of the provider in acclimating themselves to predetermined locations within the healthcare facility and the location of each of the plurality of healthcare devices 14 in the healthcare facility. In some embodiments, the system 10 approves the provider for a role in the healthcare facility, for example registered nursed, by tracking the provider's progress in acclimating themselves to predetermined locations within the healthcare facility and the location of each of the plurality of healthcare devices 14 in the healthcare facility.

As described in more detail below, a provider also uses the virtual reality device 20 to train on the various healthcare devices 14 because the learning and management system 18 includes a library of device training simulations to train the provider on operation of at least one of the plurality of healthcare devices 14 using the virtual reality device 20. For example, upon arriving at room 202 (FIG. 4), the provider virtual enters the room and locates the monitor 56 (FIG. 5). The virtual display of the monitor 56 opens at least one training simulation related to the monitor 56. The training simulation opened is based on a role of the provider, in some embodiments. For example, is the device 20 is being operated by a certified nurse assistant, the learning and management system 18 opens a training simulation in the virtual reality device 20 related to taking vitals with the monitor 56 but not documenting bedside assessments. In contrast, a registered nurse is provided a training simulation on both taking vitals and documenting bedside assessments with the device. In some embodiments, the provider's access to the at least one of the plurality of healthcare devices 14 is based on the device training simulations completed. In some embodiments, the provider's access to the at least one of the plurality of healthcare devices 14 is limited based on the device training simulations completed. For example, the certified nurse assistant's access is limited, in some embodiments. In some embodiments, the provider is granted full access to the at least one of the plurality of healthcare devices 14 based on the device training simulations completed. In some embodiments, the system 10 tracks the device training simulations completed by the provider. For example, the registered nurse is granted full access to the monitor 56 upon completion of all relevant training simulations related to the monitor 56.

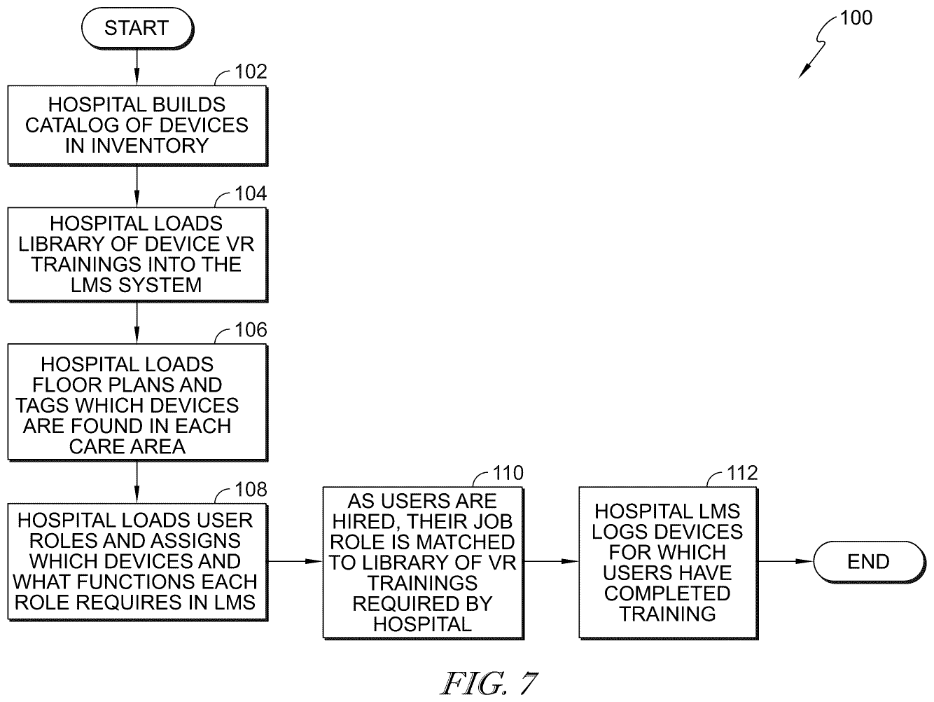

Referring now to FIG. 7, a method 100 is provided for completing training using the virtual reality training system 10. The method 100 includes building a catalog of healthcare devices 14 in inventory at the healthcare facility, at step 102. That is, upon implementing the system 10, a catalog of every healthcare device 14 in the healthcare facility is built. It will be appreciated that the catalog is updated over time to account for new devices 14 being implemented at the healthcare facility and removing old or damaged healthcare devices 14 from the inventory. In some embodiments, the catalog is temporarily updated is a device 14 is out for maintenance. In some embodiments, the catalog is updated on a schedule, for example daily, weekly, or monthly.

Virtual reality device training simulations are created for each healthcare device 14 inventoried. In some embodiments, the device training simulations are created for only selected devices. In some embodiments, the device training simulations are created by a manufacturer and uploaded upon purchase of the healthcare device 14. In some embodiments, the device training simulations are created by training personnel at the healthcare facility. In such an embodiment, the device training simulations are unique to the healthcare facility. At step 104, a library of device training simulations corresponding to the healthcare device inventory is uploaded into the learning and management system 18.

At step 106, facility data and device data is stored in the learning and management system 18. The facility data includes a map of the specific floor plan of the healthcare facility. In some embodiments, the map is supplemented with images of the healthcare facility taken with the camera system 22. In some embodiments, the map is supplemented with other images taken of the healthcare facility. The device data includes the location of each healthcare device 14 as determined by the RTLS 16. In some embodiments, the location of each device 14 is updated in real-time using the RTLS 16. The learning and management system 18 tags the location of each healthcare device 14 on the map.

At step 108, the learning and management system 18 loads provider roles and assigns healthcare devices 14 to each provider based on the provider's role. For example, a registered nurse is assigned a predetermined set of healthcare devices 14 to learn for their role as a nurse, whereas a technician is assigned a different predetermined set of healthcare devices 14 to learn for their role as a technician. In some embodiments, a provider is only assigned certain aspects of the healthcare device 14 to learn. Accordingly, is some embodiments, there are several device training simulations related to a particular healthcare device 14, but the provider is only assigned a portion of the device training simulations for that healthcare device 14.

At step 110, each provider's job role is matched to the library of virtual reality device training simulations required by the healthcare facility. The provider uses the virtual reality device 20 to navigate the virtual environment replicating the healthcare facility so that the provider can learn the layout of the facility, as well as, the location of each healthcare device 14 in the facility. Upon virtually encountering a healthcare device 14, the provider uses the virtual reality device 20 to navigate through the required device training simulations for that healthcare device 14. For example, the provider, in some embodiments, is alerted that certain device training simulations are required for a healthcare device 14, when the healthcare device 14 is virtually encountered. In other embodiments, if the provider attempts to complete a device training simulation that is not required, the provider is alerted.

The system 10 tracks which device training simulations have been completed by each provider, at step 112. Upon completing certain device training simulations, the provider is approved for using particular healthcare device 14. In some embodiments, the provider is granted limited access to a healthcare device 14 based on the device training simulations completed. In some embodiments, the provider is granted full access to the healthcare device 14 based on the device training simulations completed. If no device training simulations have been completed or a limited number of device training simulations have been completed for a particular healthcare device 14, the provider is prevented from using that healthcare device 14, in some embodiments. It will be appreciated that device training is provided for medical personnel, as well as, non-medical personnel, in some embodiments. Accordingly, the healthcare devices 14 include both medical and non-medical devices, in some embodiments.

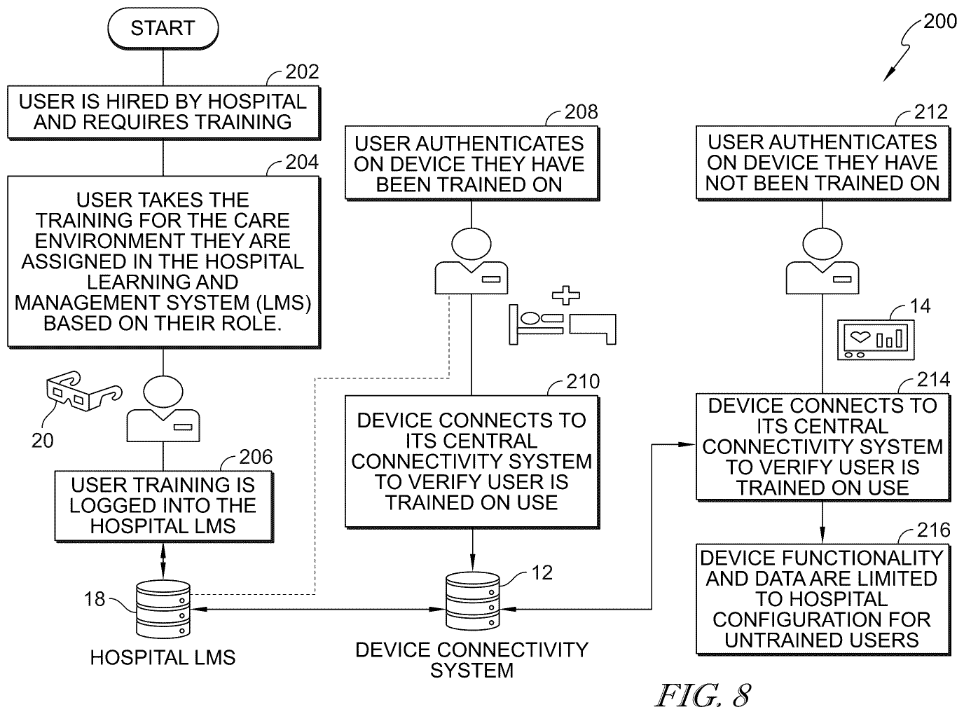

Referring not to FIG. 8, another method 200 for using the virtual reality training system 10 to authenticate a user to operate medical devices in the healthcare facility is provided. At step 202, a new provider is hire at the healthcare facility. It will be appreciated that the method 200 also applies to current providers who have changed roles or need additional training. At step 204, the provider completes the device training simulations that are assigned to that provider in the method 100 described above using the virtual reality device 20. At step 206, the provider training is logged into the learning and management system 18. That is, a list of device training simulations completed by the provider is stored or tracked in the learning and management system 18. Additionally, a list of incomplete device training simulations is also tracked in the learning and management system 18, in some embodiments.

At step 208, a provider who has completed the device training simulations for a particular healthcare device 14 attempts to authenticate use of the particular healthcare device 14. The particular device 14 connects to the central connectivity system 12, at step 210, and the authentication attempt is delivered to the central connectivity system 12 to verify if the provider is trained on the particular device 14. The central connectivity system 12 communicates with the learning and management system 18 and confirms that the provider has completed all of the device training simulations for the particular device 14 and the provider is granted full access to the particular device 14.

At step 212, a provider who has not completed all of the device training simulations for a particular healthcare device 14 attempts to authenticate use of the particular healthcare device 14. The particular device 14 connects to the central connectivity system 12, at step 214, and the authentication attempt is delivered to the central connectivity system 12 to verify whether the provider is trained on the particular device 14. The central connectivity system 12 communicates with the learning and management system 18 and confirm that the provider has not completed all of the device training simulations for the particular device 14. Accordingly, the provider is not granted full access to the particular device 14. In some embodiments, at step 216, use of the particular device 14 is limited to a configured for untrained users, wherein the functionality of the particular device 14 is limited.

The embodiments described herein use existing data points about the healthcare facility to create a personalized virtual healthcare facility, which can be used to train on a variety of different workflows and use cases. Additionally, the embodiments described herein facilitate training healthcare providers still in school, as they complete tasks in a virtual environment to get experience. The embodiments described herein are useable to complete different training modules related to a future healthcare facility's specific goals. Rather than a generic layout/hospital tasks, the embodiments described herein use the healthcare facility's specific layout and data points to create a personalized training experience. The embodiments described herein include a library of devices that the healthcare facility has purchased. The virtual reality training modules for these devices are loaded into the learning & management system so that the correct job roles are trained to the correct devices. The embodiments described herein are flexible to provide the right training for the correct scope of practice for the user. For example, a certified nursing assistant can take vitals with a vital signs device but not document bedside assessments. In contrast, a registered nurse can both take vitals and document bedside assessments with the device. The embodiments described herein confirm that a user authenticating a device has been trained in the virtual reality system. The device is configurable to the healthcare facility's preference for the functionality available to the user or how the data is treated on visualization tools or charting into the electronic medical record.

Any theory, mechanism of operation, proof, or finding stated herein is meant to further enhance understanding of principles of the present disclosure and is not intended to make the present disclosure in any way dependent upon such theory, mechanism of operation, illustrative embodiment, proof, or finding. It should be understood that while the use of the word preferable, preferably or preferred in the description above indicates that the feature so described can be more desirable, it nonetheless cannot be necessary and embodiments lacking the same can be contemplated as within the scope of the disclosure, that scope being defined by the claims that follow.

When terms of degree such as “generally,” “substantially,” and “about” are used herein in connection with a numerical value or a qualitative term susceptible to a numerical measurement, it is contemplated that an amount that is plus or minus 10 percent, and possibly up to plus or minus 20 percent, of the numerical value, is covered by such language, unless specifically noted otherwise, to at least account for manufacturing tolerances. Otherwise, a suitable definition for “generally,” “substantially,” and “about” is largely, but not necessarily wholly, the term specified.

In reading the claims it is intended that when words such as “a,” “an,” “at least one,” “at least a portion” are used there is no intention to limit the claim to only one item unless specifically stated to the contrary in the claim. When the language “at least a portion” and/or “a portion” is used, the item can include a portion and/or the entire item unless specifically stated to the contrary.

It should be understood that only selected embodiments have been shown and described and that all possible alternatives, modifications, aspects, combinations, principles, variations, and equivalents that come within the spirit of the disclosure as defined herein or by any of the following claims are desired to be protected. While embodiments of the disclosure have been illustrated and described in detail in the drawings and foregoing description, the same are to be considered as illustrative and not intended to be exhaustive or to limit the disclosure to the precise forms disclosed. Additional alternatives, modifications and variations can be apparent to those skilled in the art. Also, while multiple inventive aspects and principles have been presented, they need not be utilized in combination, and many combinations of aspects and principles are possible in light of the various embodiments provided above.

1. A virtual reality training system for a healthcare facility, the system comprising:
a real time locating system for locating each of a plurality of healthcare devices within a healthcare facility, wherein the plurality of healthcare devices belong within a predetermined category of healthcare devices,
a learning and management system for storing device data related to a location of each of the plurality of healthcare devices within the predetermined category, the learning and management system further storing facility data including a map of the healthcare facility, and
a virtual reality application configured for training providers in the healthcare facility, the virtual reality application configured for displaying images of the healthcare facility based on the facility data, wherein the images of the healthcare facility include representations of each of the plurality of healthcare devices within the predetermined category at the respective location in the healthcare facility based on the device data, wherein a provider utilizes the virtual reality application to acclimate themselves to the location of each of the plurality of healthcare devices within the predetermined category.

2. The system of claim 1, wherein the predetermined category of healthcare devices includes healthcare devices related to a role of the provider in the healthcare facility.

3. The system of claim 1, wherein the predetermined category of healthcare devices includes healthcare devices in storage at the healthcare facility.

4. The system of claim 1, wherein the predetermined category of healthcare devices includes healthcare devices related to a type of training module.

5. The system of claim 1, wherein the virtual reality application is operated on a virtual reality headset worn by the provider.

6. The system of claim 1, wherein the provider utilizes the virtual reality application to acclimate themselves to predetermined locations within the healthcare facility based on a role of the provider within the healthcare facility.

7. The system of claim 6, wherein the system tracks a progress of the provider in acclimating themselves to the predetermined locations.

8. The system of claim 7, wherein the system approves the provider for the role based on the progress of the provider.

9. The system of claim 1, wherein the real time locating system updates the location of one of the plurality of healthcare devices in response to the one of the plurality of healthcare devices being moved.

10. The system of claim 1, wherein the device data related to the location of each of the plurality of healthcare devices is updated in response to one of the plurality of healthcare devices being taken offline.

11. The system of claim 1, wherein the device data related to the location of each of the plurality of healthcare devices is updated in response to a new healthcare device being implemented at the healthcare facility.

12. The system of claim 1, wherein the learning and management system includes a library of device training simulations to train the provider on operation of at least one of the plurality of healthcare devices using the virtual reality application.

13. The system of claim 12, wherein the provider's access to the at least one of the plurality of healthcare devices is based on the device training simulations completed.

14. The system of claim 13, wherein the provider's access to the at least one of the plurality of healthcare devices is limited based on the device training simulations completed.

15. The system of claim 13, wherein the provider is granted full access to the at least one of the plurality of healthcare devices based on the device training simulations completed.

16. The system of claim 12, wherein the system tracks the device training simulations completed by the provider.

17. The system of claim 1, wherein the provider includes a medical provider.

18. The system of claim 1, wherein the provider includes at least one of maintenance personnel, cleaning personnel, and administration personnel.

19. The system of claim 1, wherein the plurality of healthcare devices includes a plurality of medical devices.

20. The system of claim 1, wherein the plurality of healthcare devices includes a plurality of non-medical devices.
